(19) United States
(12) Patent Application Publication
(10) Pub. No.: US 2007/0293546 A1
(43) Pub. Date: Dec. 20, 2007

(54) PREPARATION OF ROSIGLITAZONE AND ITS SALTS

(76) Inventors: Srinivasula Reddy Maddula, Guntur District (IN); Kushal Surajmal Manudhane, Hyderabad (IN); Sridhar Thota, Hyderabad (IN); Arvind Mallinath Lali, Mumbai (IN); Sandeep Bhaskar Kale, Mumbai (IN); Venkata Naga Brahmeswara Rao Mandava, Hyderabad (IN); Radhakrishna Singam Setty, Hyderabad (IN); Sandeep Mohonty, Bhubaneswar (IN); Jaga Gangaraju Pericherla, West Godavari (IN); Manoj Ramesh Kharkar, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/763,775

(22) Filed: Jun. 15, 2007

Related U.S. Application Data
(60) Provisional application No. 60/821,451, filed on Aug. 4, 2006.
(60) Provisional application No. 60/826,793, filed on Sep. 25, 2006.

(30) Foreign Application Priority Data
Jun. 15, 2006 (IN) 1036/CHE/2006
Jul. 4, 2006 (IN) 1168/CHE/2006

(51) Int. Cl. C07D 417/02; A61K 31/4439

(52) U.S. Cl. 514/342; 546/269.7

(57) ABSTRACT

The present invention relates to rosiglitazone and its pharmaceutically acceptable salts free of at least one of the process related impurities, in particular the dehydro and the succinic acid impurities of rosiglitazone, wherein said impurities are present in an amount of about 05 mg to not more than about 15 mg, and processes for their preparation.

### INTRODUCTION TO THE INVENTION

The present invention relates to a process for preparation of rosiglitazone and its pharmaceutically acceptable salts. In particular, the present invention relates to rosiglitazone and its pharmaceutically acceptable salts free of at least one of the dehydro and the succinic acid impurities of rosiglitazone and processes for their preparation.

### BACKGROUND OF THE INVENTION

Rosiglitazone maleate is described chemically as (±)-5-[[4-[2-(methyl-2-pyridinylamino)ethoxy]phenyl]methyl]-2,4-thiazolidinedione maleate (1:1) and is structurally represented by Formula I.

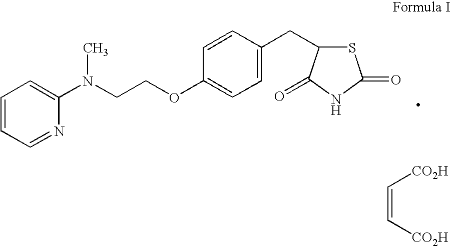

Rosiglitazone maleate is used as an adjunct to diet and exercise to improve glycemic control in patients with Type 2 diabetes mellitus. It is marketed as AVANDIA® tablets comprising 2 mg, 4 mg or 8 mg rosiglitazone maleate.

European Patent No. EP0306228 discloses substituted thiazolidinedione derivatives including rosiglitazone having hypoglycemic and hypolipidemic activity. PCT Application Publication No. WO 94/05659 discloses salts of rosiglitazone and its derivatives and also describes maleate salt as the preferred salt. US Application Publication No. 2002115866 discloses the preparation of rosiglitazone and its maleate salt. PCT Application Publication No. WO 2005/021541 discloses processes for the preparation of rosiglitazone, its polymorphic forms, pharmaceutical compositions and therapeutic uses. GB 2410948 and AU 2002352390 disclose thiazolidinedione phosphate and citrate as a novel salt of rosiglitazone.

The synthesis of rosiglitazone involves many synthetic steps in which undesired products are obtained. Therefore, the final product can be contaminated not only with the undesired products derived from the last synthetic step of the process but also with compounds that were formed in previous steps. Rosiglitazone or its salts when prepared according to the processes disclosed in the above documents was found to contain impurities such as the dehydro impurity represented by Formula III, which is chemically described as 5-[4-[2-[(N-methyl)-N-(2-pyridinyl)amino]ethoxy]benzylidine]-2,4-thiazolidinedione (hereinafter referred to as “dehydro impurity of rosiglitazone”).

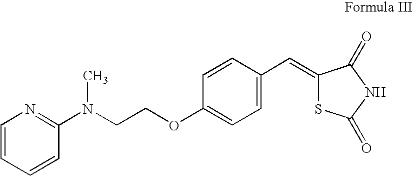

Regulatory authorities worldwide require that drug manufacturers isolate, identify and characterize the impurities in their products. Moreover, it is required to control the levels of these impurities in the final drug compound obtained by the manufacturing process and to ensure that the impurities are present in the lowest possible levels. Thus, process related impurities should be removed from the final product in order to meet the ICH specifications for purity.

None of the documents cited above disclose a process for the purification of rosiglitazone and its salts wherein rosiglitazone is free of its process related impurities. Hence, there is a need for a purification method for rosiglitazone that uses a simple and commercially viable process while achieving the desired purity.

The present invention provides a process for the purification of rosiglitazone and its salts to give a product that is substantially free of its process related impurities and also free of residual organic solvents. The processes of the present invention can be practiced on an industrial scale, and also can be carried out without sacrifice of overall yield based on the starting materials employed.

### SUMMARY OF THE INVENTION

Thus, it is an object of the present invention to provide a process for the purification of rosiglitazone and its pharmaceutically acceptable salts. In particular, the present invention provides a process for the preparation of rosiglitazone and its pharmaceutically acceptable salts free of at least one of the dehydro and the succinic acid impurities.

In one aspect the present invention provides a process for the preparation of rosiglitazone maleate of Formula I

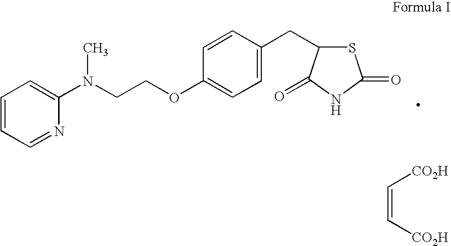

wherein rosiglitazone succinic acid impurity of Formula II

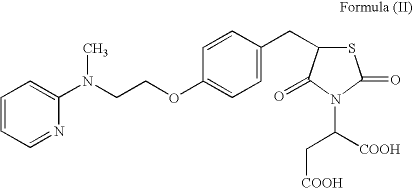

is present in an amount of not more than 0.15 area percent by HPLC, which process comprises:
(a) heating a reaction mass containing rosiglitazone maleate freebase and a ketone to about 60° C.;
(b) adding maleic acid dissolved in an organic solvent followed by addition of activated carbon;
(c) isolating rosiglitazone maleate;
(d) drying at temperatures of from about 25° C. to about 40° C. with or without vacuum and with or without inert atmosphere, to afford the desired rosiglitazone maleate compound of desired purity.

In another aspect the present invention provides a process for the purification of rosiglitazone and its salts to substantially remove the dehydro impurity having Formula III,

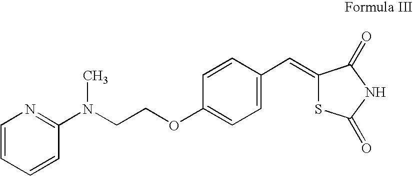

which process comprises:
(a) adsorbing rosiglitazone onto an adsorbent resin, by contacting a resin with a rosiglitazone solution; and
(b) eluting purified rosiglitazone from a resin with a solvent comprising 5% v/v to 70% v/v linear gradient of methanol in phosphate buffer.

Another object of the present invention is to provide a composition comprising rosiglitazone maleate substantially free of at least one of the dehydro and the succinic acid impurities.

Thus, in one aspect the present invention provides a composition comprising:
(a) a compound which is rosiglitazone maleate of Formula I present in the amount of about 05 mg to about 15 mg; and
(b) a compound which is rosiglitazone succinate of Formula II present in an amount of from about 0.001% to not more than about 0.05 area percent by HPLC

In another aspect the present invention provides a composition comprising:
(a) a compound which is rosiglitazone maleate of Formula I, present in the amount of about 05 mg to about 15 mg; and
(b) a compound which is the dehydro impurity of rosiglitazone having a Formula III present in an amount of from about 0.001% to about not more than 0.10 area percent.

In yet another aspect the present invention provides a composition comprising:
(a) a compound which is rosiglitazone maleate of Formula I present in the amount of about 05 mg to about 15 mg;
(b) a compound which is rosiglitazone succinate of Formula II, present in an amount of about 0.001% to not more than about 0.05 area percent by HPLC; and
(c) a compound which is the dehydro impurity of rosiglitazone having a Formula III present in an amount about 0.001% to not more than about 0.10 are percent.

In another aspect the present invention provides a composition wherein said rosiglitazone maleate of Formula I is present in the amount of about 1 mg to about 10 mg.

In another aspect the present invention provides a composition wherein said rosiglitazone succinate impurity of Formula II, is present in an amount of about 0.001% to about 0.10 area percent by HPLC.

Still another aspect of the present invention provides 2-[5-[[4-[2-(methyl-2-pyridinylamino)ethoxy]phenyl]methyl]-2,4-thiazolidinedione] succinic acid of Formula II

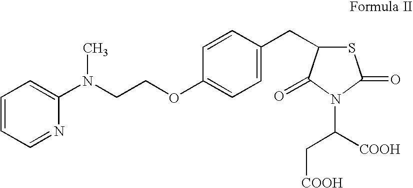

and this impurity is an intermediate for the preparation of rosiglitazone which is carried to the final product due to incomplete reduction in the final stage.

The rosiglitazone succinic acid impurity is formed due to the contact of rosiglitazone maleate with moisture. Rosiglitazone maleate is sensitive to moisture and high temperature as presence of a combination of the two leads to an increase in the percentage of the succinic acid impurity of rosiglitazone.

Rosiglitazone or any of the pharmaceutically acceptable salts of rosiglitazone prepared in accordance with the present invention contain less than about 05 mg and no more than about 15 mg of the corresponding impurities like rosiglitazone dehydro impurity and the rosiglitazone succinic acid impurities as characterized by a high performance liquid chromatography (“HPLC”) chromatogram obtained from a mixture comprising the desired compound and one or more of the said impurities. Preferably, said process related impurities are about less than 0.5%, or less than about 0.10%. The percentage here refers to the area-% of the peaks representing the said impurities.

In one embodiment, the present invention provides a composition wherein rosiglitazone maleate of Formula I which is substantially free of at least one of the dehydro and the succinic acid impurities, comprising:
(a) a compound which is rosiglitazone maleate of Formula I present in the amount of about 05 mg to about 15 mg or from about 1 mg to about 10 mg;
(b) rosiglitazone succinate of Formula II, present in an amount of from about 0.001% to not more than about 0.10 area percent by HPLC; and/or
(c) the dehydro impurity of rosiglitazone having a Formula III present in an amount of more than about 0.001% and not more than about 0.10.

In another embodiment, the present invention provides a composition composition wherein rosiglitazone maleate of Formula I which is substantially free of at least one of the dehydro and the succinic acid impurities, comprising:

useful as a reference standard for determining the purity of rosiglitazone by HPLC, and a process for its preparation.

In another aspect, the present invention provides a process for preparing the rosiglitazone succinic acid of Formula II, which process comprises heating rosiglitazone maleate in water.

Another object of the present invention provides a pharmaceutical composition that includes a therapeutically effective amount of pure rosiglitazone or its salts prepared according to the processes of the present invention and one or more pharmaceutically acceptable carriers, excipients or diluents.

### DETAILED DESCRIPTION OF THE INVENTION

The present invention relates to a process for purification of rosiglitazone and its pharmaceutically acceptable salts. In particular, the present invention relates to rosiglitazone and its pharmaceutically acceptable salts free of at least one of the dehydro and the succinic acid impurities of rosiglitazone and processes for its preparation.

Unless otherwise specifically mentioned, the term rosiglitazone mentioned hereinafter is intended to cover the pharmaceutically acceptable salts and solvates, hydrates, and all crystalline and amorphous forms of rosiglitazone.

The dehydro impurity of rosiglitazone is chemically 5-[4-[2-[(N-methyl)-N-(2-pyridinyl)amino]ethoxy]benzylidine]-2,4-thiazolidinedione represented by Formula III

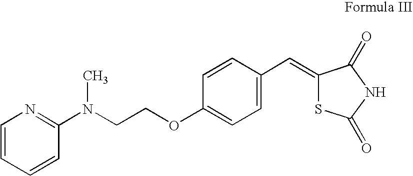

(a) a compound which is rosiglitazone maleate of Formula I present in the amount of about 05 mg to about 15 mg or from about 1 mg to about 10 mg;
(b) a compound which is rosiglitazone succinate of Formula II present in an amount of from about 0.001% to not more than about 0.05 area percent by HPLC; and/or
(c) the dehydro impurity of rosiglitazone having a Formula III present in an amount of more than about 0.001% and not more than about 0.10.

Another embodiment of the present invention provides a process for the purification of rosiglitazone to substantially remove the dehydro impurity using adsorption chromatography.

Even though purification by crystallization and by using adsorption chromatography are known to be the simplest processes that can be used for purification of organic compounds, many of the impurities are hard to remove as they co-crystallize with rosiglitazone or its salts. The right choice of solvents for crystallization during recrystallization and the right choice of solvent system for elution in column purification play a major role in removing the undesired impurities from the compound and therefore purifying it. The solvent of choice should effectively remove the impurity without sacrificing the yield.

In an embodiment, the process for purification of rosiglitazone using adsorption chromatography comprises of:
a) providing a solution of crude rosiglitazone;
b) adjusting the pH of the sample obtained in step a);
c) adding the rosiglitazone crude sample onto an adsorption column bed;
d) eluting with an eluting solution;
e) regeneration/cleaning-in-place of the column.

Step a) providing a solution of crude rosiglitazone: Rosiglitazone obtained using any of the processes described in the art can be purified using the purification process of the present invention. The solution of crude rosiglitazone may be obtained by suspending rosiglitazone in a suitable solvent, or such a mixture may be obtained directly from a reaction in which rosiglitazone is formed. The crude rosiglitazone can typically either be an aqueous reaction mixture obtained after the synthesis of rosiglitazone and before extraction or it may be a crude organic extract obtained after the extraction of the product into an organic layer containing most of the process related impurities. The crude rosiglitazone base may contain, in addition to the rosiglitazone base, quantities of dehydro impurity of rosiglitazone, and other impurities.

The organic solvents which can be used to prepare the solution of rosiglitazone are selected from tetrahydrofuran (THF), acetone, acetonitrile (ACN), methanol, ethanol, n-butanol, n-propanol, iso propanol, esters (e.g. ethyl acetate), and dipolar aprotic solvents such as dimethylformamide (DMF).

Step b) adjusting the pH: The crude rosiglitazone sample either in the form of aqueous reaction mixture, or crude organic extract is further processed to acquire the required pH. When treating the reaction mixture containing rosiglitazone and the impurities with a resin, the operation conditions should not be damaging to rosiglitazone, e.g. strongly basic acidic (pH>7) conditions and high temperatures (temperature>70° C.) should be avoided. The pH of rosiglitazone containing liquid is adjusted to a suitable value, for example about 2 to about 7, or from about 2 to about 4.

The molarity of the rosiglitazone solution may be adjusted to a suitable value, for example about 0.01 M to about 1.0 M, or about 0.05 M to about 0.8 M, or about 0.1 M to about 0.5 M, with a suitable salt.

The conductivity of the rosiglitazone solution may be adjusted to a suitable value, for example about 1 to 50 milliSiemens/centimeter (“mS/cm”) or about 10 to about 40 mS/cm or about 12 to about 35 mS/cm with suitable salt, acid and/or alkali. “mS/cm” refers to the unit of conductivity of solution.

Rosiglitazone solution having the required pH can be obtained by dissolving rosiglitazone in an aqueous buffer solution. The amount of water used is to be sufficient to dissolve the rosiglitazone or its salt. The pH of the aqueous buffer solution may range from about 2 to about 5. The pH of the solution of rosiglitazone in the buffer may be further adjusted using any method known in the art. Typically, the pH of the aqueous solution may be adjusted using mineral or organic acid in sufficient amount to achieve the required pH of about 6 to about 8.

The substances that are used for adjustment of pH are typically chosen from substances that are pharmaceutically acceptable. The bases used for the adjustment of pH of the aqueous buffer solution include, but are not limited to, potassium dihydrogen phosphate, potassium phosphate, sodium phosphate, sodium acetate, potassium acetate, sodium chloride, monoammonium phosphate, diammonium phosphate, sodium carbonate, and pH may be adjusted with substances including but not limited to suitable diluted mineral or organic acids and/or alkaline substances. Acids like phosphoric acid, hydrochloric acid, sulfuric acid, acetic acid, citric acid, nitric acid, and alkaline substances such as ammonia, sodium hydroxide, potassium hydroxide, calcium hydroxide and the like are useful.

Step c) addition of the rosiglitazone crude sample to an adsorption column bed: The rosiglitazone crude sample can either be in the form of a solution or a crude extract, or residue. The sample prepared above is adsorbed onto a suitable amount (for example about 1 to about 50 parts by volume per part by volume of the crude rosiglitazone containing liquid) of the resin. Crude rosiglitazone containing impurities in the form of solution or crude is brought into contact with a hydrophobic natural or synthetic polymer based resin. The contact can be made either by pouring the solution or a crude on the resin contained in a column, or by other methods known to one skilled in the art.

Generally, the adsorption column bed includes resins such as polymeric adsorbents with highly porous structures whose internal surfaces can adsorb and desorb a wide variety of different species depending on the environment in which they are used. In this case, with a polar solvent such as water, the polymeric adsorbents exhibit non-polar or hydrophobic behavior and may adsorb organic species that are sparingly soluble.

The resins used in the adsorption steps can be identical or different. Selection of resin depends upon the properties of the resin (pore size, grain size, surface area, polarity of the surface and solubility index), type of material to be purified, level and nature of impurities present, type of medium used for sample preparation, mobile phase used. Other factors that play role in the selection are chromatographic conditions like temperature, flow rate, pH and gradient volume.

Resins which can be used for the purpose of the present invention include but are not limited to commercially available resins like those manufactured by Rohm and Haas, Philadelphia Pa., and Thermax such as AMBERLITE_XAD_{—}4, XAD_{—}7, XAD_{—}16, XAD_{—}1 6HP, XAD_{—}761, and XAO_{—}1180 and ADS-600, 400. Other resins suitable for the process of the invention include those manufactured and sold by Mitsubishi Kasei Corporation, Japan, such as DAION_HP 10, DAION_HP 20, DAION_HP 21, DAION_HP 30, DAION_HP 40, DAION_HP 50, DAION_SP 800, DAION_SP 825, DAION_SP 850, DAION_SP 875, DAION_SP 205, DAION_SP 207, DAION_HP1MG, and DAION_HP2MG, or SEPABEADS SP207, SP825, SP700, SP207SS, SP850, SP20SS (Mitsubishi, Japan), SOURCE 5 RPC, 15 RPC, Phenyl Sepharose 6 FF, HP, high substitution, Butyl and Octyl Sepharose 4FF (GE Biosciences) or Amberlite XAD-2, XAD-4, XAD-7, XAD-8 or XAD-9 (products of Rohm and Haas, U.S.A.), can be employed with good results.

The resins which can be used also include a divinylbenzene-styrene copolymer or, a copolymer of divinylbenzene, styrene and other derivatives of these having aliphatic and/or aromatic moieties comprising from 2 to 18 carbon atoms, or having substituted halogen atoms from chloride, fluoride or bromide, or a copolymer of divinylbenzene and styrene with surface grafted moieties that are aliphatic or aromatic containing 2 or more carbon atoms and/or having substituted halogen atoms from chlorine, fluorine or bromine, or its combination with other polymers, prepared by cross-linking of monomers containing ionic or affinity group interactions.

The adsorbent resin may be filled into any suitable container or reaction vessel such as for example a filtration funnel or a cylindrical vessel or the like. The size of the container may be chosen based on the amount of the resin to be contained therein and the batch size of rosiglitazone to be purified.

The “interacting group(s)” are the groups present on the surface of resin. These groups can have different interactions with different solutes to achieve separation. These are chemical moieties which are a part of base matrix or attached to the base matrix of the resin by known activation chemistry and interact to the adsorbing molecule by any known type of reversible bonding mechanism such as but not limited to hydrophobic, ionic, co-ordinate or mixed mode of interactions. The interacting groups of the present invention may be the part of base matrix or may be grafted on the matrix by the known activation chemistry to give the desired characteristics such as hydrophobicity, group density, spatial orientation, surface area, porosity, pore structure, particle size, and pore radius to the resin.

The technical realization of adsorption may vary depending on the equipment chosen. The operation may be carried out batch wise or in a continuous fashion. In a batch wise operation, the resin can be admixed with the reaction mixture, and can be separated carrying the adsorbed materials, with conventional techniques, e.g. sedimentation, filtration.

According to one embodiment, the adsorption is performed with a packed bed column chromatographic or fluidized bed chromatographic technique, which comprises filling the column with a suitable adsorbent in a suitable column and passing the rosiglitazone containing liquid through the filled pre-equilibrated resin (resin column is equilibrated with the mobile phase). The packed column is equilibrated before loading rosiglitazone with the mobile phase containing 1 to 50% v/v of water in organic solvent. The proportion of organic solvent in the equilibrating mobile phase is in the range of 50 to 99% v/v depending upon the type of organic solvent.

For the continuous mode of operation packed bed, simulated moving bed (SMB), improved simulated moving bed (ISMB), centrifugal chromatography, continuous annular chromatography or any combination thereof can be used. In case of batch system a stirred tank or agitated tank can be used.

In the present invention a suitable amount (for example about 1 to about 50% v/v of the rosiglitazone containing liquid) of the adsorbent filled into the column. The feed is pumped into the column at the rate of 50 to 500 cm/hr linear flow velocity or more preferably 100 to 300 cm/hr linear flow velocity and the elution fraction corresponding to the pure rosiglitazone is collected. The adsorbed resin column is washed with mobile phase of same or different composition as that of equilibration mobile phase followed by gradient elution to resolve the rosiglitazone from impurities. The temperature used in the adsorption operation may range from about 10° C. to about 50° C., or from about 20° C. to about 30° C.

The batch method can also be performed by passing the rosiglitazone containing liquid through a layer of the filled pre-equilibrated (equilibrated with buffer), adsorbent under pH and temperature conditions similar to those shown in the case of the column chromatographic method.

It has been surprisingly found that the dehydro impurity is extensively bound to the silica gel, while rosiglitazone can be substantially completely eluted with a solvent, thereby avoiding the need to change the polarity of the solvents used for elution.

Step d) eluting of rosiglitazone: Eluting rosiglitazone or rosiglitazone salt with an eluting solution is carried out using techniques known to one of ordinary skill in the art. The eluting solution is pumped into the column, using a pump and the fraction is collected after discarding void volume which is the dead volume of the column fraction. After loading, the column is washed with a buffer of the same or different molarity as that which is used for feed preparation. The collection is continued during washing until 1 to 15 column volumes or 8 to 12 column volumes of total column volume fraction is collected. After collection of the active pharmaceutical ingredient (rosiglitazone) the column is flushed with 1 to 3 column volumes of demineralized water.

Column volume refers to the volume in ml or liter and is equivalent to volume of resin material filled in column. For example, a column with dimensions 4.0 cm×100 cm, has a volume which is calculated as: cross-sectional area×L=12.56×100=1256 ml or 1.256 L, hence the column volume for a column with the above mentioned dimensions is: (CV)=1.256 (same volume of resin can be filled in the 4 cm×100 cm column).

According to one embodiment, rosiglitazone is resolved from its related impurities not only by the stepwise or linear gradient of mobile phases but also by the isocratic elution with mobile phase. If gradient elution is used the gradient volume may be 0.5 to 10.0 column volumes, preferably 1.0 to 5.0 column volumes and most preferably 1.5 to 2.5 column volumes.

The mobile phase contains organic modifiers such as but not limited to alcohols such as for example methanol, ethanol, isopropanol, butanol and the like; nitrile solvents such as for example acetonitrile and the like; chlorinated organic solvents such as for example chloroform, dichloromethane and the like; toluene; esters such as for example butyl acetate, ethyl acetate and the like, ketones such as for example acetone, methyl ethyl ketone and the like; and any suitable combination of one or more than one of these solvents. Water may also be combined with these solvents to adjust the polarity as required. Other solvents may also be used as required without limitation.

The mobile phase used may also contain ion-pairing agents or polarity modifiers such as but not limited to phosphoric acid, acetic acid, pentane sulphonic acid, trifluoro acetic acid, tetrahydrofuran, triethylamine and any suitable combination of one or more than one thereof. The concentration of ion-pairing agent in the mobile phase ranges from about 0.001% v/v to about 2.5% v/v depending upon the type of ion-pairing agent selected. Other ion pairing agents or polarity modifiers that are useful in the invention are citrate buffer, phosphate buffer, acetate buffer, phosphate-citrate buffer, hexane sulphonic acid, heptane sulphonic acid and the like. Other polarity modifiers or ion-pairing agents may also be used without limitation. The mobile phase may contain about 1% v/v to about 50% v/v of water. If water is used in the mobile phase then the mobile phases used in the gradient chromatography can be miscible with each other.

Suitable solvents for elution include, without limitation, buffer solution used for dissolution of crude rosiglitazone, esters such as ethyl acetate and n-butyl acetate; ketones such as acetone; alcohols such as isopropanol, methanol, and ethanol; hydrocarbons such as cyclohexane; and the like. Mixtures of such solvents, in various proportions, can also be used. The temperature of the solvent used for elution can range from about 5° C. to about 80° C. The appropriate temperature to be used will be determined based on the solvent or solvent mixture that is used to achieve optimal separation. The flow of the solvent can be continuous or in lots and the quantity of solvent can range from about 2 to about 20 times the volume of the solution containing the crude rosiglitazone.

Suitably, the flow rate (relative to the cross-sectional area) is less than about 100 ml/minute, or less than about 50 ml/minute. Lower elution rates increase the time, but improve the separation efficiency. The eluent flowing out of the bed of sorption resin (i.e. the eluent) is collected in one or more fractions, using separation methods that depend on preferential retention of chemical species on a stationary phase (e.g., a static bed), such as for example, chromatography. An inorganic acid, such as phosphoric acid, may be added to the eluent.

In the present invention the term “gradient volume” means the volume of mobile phase in which the final strength of the eluting mobile phase is achieved.

The term “related impurities” means the impurities generated during the synthesis or during processing before the chromatographic step and are structurally related to the active pharmaceutical ingredient.

In the gradient elution chromatography, if carried out under optimally selected conditions, a purified rosiglitazone, which is essentially devoid of most of impurities including dehydro rosiglitazone, can be obtained. The presence of dehydro rosiglitazone and other impurities can be determined by quantitative techniques such as HPLC.

Rosiglitazone separated from impurities and therefore having a reduced level of impurities can be isolated from eluent by any conventional means such as for example, extractions lyophilization, evaporation, or by addition of an anti-solvent. Examples of anti-solvents which can be used include water, alkanes and cycloalkanes.

A preferred method of isolation includes concentration of the main fraction at 70° C. or less, preferably 60° C. or less, preferably at pressure of 760 mm Hg, to about 50% of its initial volume, whereby crystals of product are obtained. The rosiglitazone obtained in the required fractions has a low amount of the process related impurities especially, the dehydro impurity. The total yield is between 90 to 100% with respect to the feed content of rosiglitazone.

The present invention provides rosiglitazone containing less than about 1500 ppm, or less than about 1000 ppm, or less than about 500 ppm of the dehydro impurity. These amounts can be expressed, respectively, as less than about 0.15 area-%, less than about 0.1 area-%, and less than about 0.05 area-% in an analysis by HPLC.

Step e) regeneration of the column: The column is regenerated at rate of 100 to 500 cm/hr linear flow velocity with 4 to 15 column volumes of suitable concentrations of ordinary agents, including lower aliphatic alcohols such as methanol, ethanol and isopropanol; ketones such as acetone, esters such as ethyl acetate, butyl acetate; acids, for example inorganic acids such as phosphoric acid, sulfuric acid, and hydrochloric acid and organic aliphatic carboxylic acids such as acetic acid, citric acid; alkalies such as sodium hydroxide, potassium hydroxide, calcium hydroxide, ammonium hydroxide. The solution of acid or alkali used in the regeneration may be not less than 0.5M solution. Suitable combination of one or more or the above may be used for effective regeneration.

The regenerant mobile phase used for cleaning the resin column can be properly selected depending upon the types and amounts of impurities bound onto the adsorbent, and the type of adsorbent resin. Aqueous solutions of lower aliphatic alcohols may be more useful. Solvents such as hydrous alcohols having suitable alcohol concentration such as 100% methanol, 50-100% ethanol, and 20-50% isopropanol, and the like can be used. The regeneration operation can be carried out at temperatures of the range of about 20° C. to about 80° C., or from about 30° C. to about 50° C.

Organic solvent may be flushed out of the column with 2 to 5 column volumes of demineralized water before starting equilibration of column with an equilibration buffer. The equilibration buffer may be same as the feed and wash buffer or a higher molarity prepared by using pharmaceutically acceptable salts or acids as mentioned above.

The process of the present invention is accomplished by simple gradient elution chromatography for resolution of two structurally related molecules using reusable adsorbent resins having natural or synthetic base matrix capable of interacting with the impurities due to a group which may be a part of the base matrix or grafted onto the base matrix by known activation chemistry.

Rosiglitazone obtained by the above process can be converted to its salts by processes known in the art.

In another embodiment the present invention provides a process for the preparation of rosiglitazone maleate substantially free of the succinic acid impurity comprising:
a) providing a solution of rosiglitazone in a suitable solvent;
b) adding maleic acid to the solution obtained in step (a);
c) isolating rosiglitazone maleate from the solution of step b);
d) drying the solid obtained in step c) under controlled conditions.

Step a) providing a solution of rosiglitazone in a suitable solvent: Rosiglitazone for the purpose of conversion to its maleate salt may be prepared according to the processes described in the art, or may be obtained by a process described above. The solution of rosiglitazone may be obtained by dissolving rosiglitazone in a suitable solvent, or such a solution may be obtained directly from a reaction in which rosiglitazone is formed. When the solution is prepared by dissolving rosiglitazone in a solvent, any form of rosiglitazone such as any crystalline or amorphous form including any salts, solvates and hydrates may be utilized for preparing the solution.

Suitable solvents which can be used for dissolving rosiglitazone include but are not limited to alcoholic solvents like methanol, ethanol, isopropyl alcohol and the like, ketonic solvents such as acetone, ethylmethyl ketone, methyl isobutyl ketone and the like; hydrocarbon solvents such as toluene, xylene and the like; nitrile solvents such as acetonitrile, propionitrile and the like; or mixtures thereof.

The dissolution temperatures can range from about 20° C. to 120° C. depending on the solvent used for dissolution. Any other temperature is also acceptable as long as the stability of rosiglitazone is not compromised and a clear solution is obtained. The quantity of solvent used for dissolution depends on the solvent and the dissolution temperature adopted. The concentration of rosiglitazone in the solution may generally range from about 0.1 to about 10 g/ml in the solvent.

Step b) adding maleic acid: Maleic acid can be added to the solution either at the dissolution temperatures or after cooling the solution to lower temperatures. Maleic acid can be added in the form of a solution in an organic solvent, or it can be added directly. Suitably, solutions contain about 5% to 50%, or about 10% to 20%, (w/v) of maleic acid. Suitably, ketonic solvents like acetone, butanone, methyl isobutyl ketone, and the like are used for the preparation of the solution of maleic acid.

The solution obtained after addition of maleic acid can be optionally treated with activated charcoal to enhance the color of the compound followed by filtration through a medium such as through a flux calcined diatomaceous earth (Hyflow) bed to remove the carbon. The carbon treatment can be given either at the dissolution temperatures or after cooling the solution to lower temperatures.

Step c) isolation of rosiglitazone maleate: For isolation of the product, the reaction mass may be maintained at temperatures lower than the dissolution temperatures, such as for example about 10° C. to about 25° C., for a period of time as required for a more complete isolation of the product. The exact cooling temperature and time required for complete isolation will depend on parameters such as concentration and temperature of the solution or slurry.

Optionally isolation of the solid may be initiated by methods such as cooling, seeding, partial removal of the solvent from the solution, such as by adding an anti-solvent to the solution or a combination thereof. The isolated solid is recovered from the final mixture, with or without cooling below the operating temperature, by various techniques such as filtration by gravity, or by suction, centrifugation, and the like. The isolated crystals may carry a small proportion of occluded mother liquor. If desired the crystals can be washed on the filter with a solvent.

Step d) drying: Drying conditions are to be controlled to reduce the percentage of the succinic acid impurity in the final product.

Rosiglitazone maleate when subjected to heating for a prolonged time undergoes degradation and the percentage of the succinic acid impurity is increased. Optimized drying conditions, reduce the formation of the succinic acid impurity and also give the residual solvent content within limits of the ICH guidelines.

Suitable techniques which can be used for drying include tray drying, vacuum oven drying, air oven drying, or using a fluidized bed drier, spin flash dryer, flash dryer and the like. The drying can be carried out at temperatures ranging from about 25° C. to about 40° C. The drying can be carried out for any desired time periods, preferably about 1 to about 30 hours or about 5 to 15 hours.

Optionally, the solid obtained may be further purified by recrystallization in a suitable solvent. Recrystallization involves providing a solution of rosiglitazone maleate in a suitable solvent and then crystallizing the solid from the solution. Suitable solvents which can be used include, but are not limited to: ketonic solvents like acetone, methyol isobutyl ketone, butanone, and the like; hydrocarbons such as toluene, xylene, n-heptane, cyclohexane, n-hexane and the like; nitriles such as acetonitrile, propionitrile and the like; halogenated hydrocarbons such as dichloromethane, 1,2-dichloroethane, chloroform, carbon tetrachloride and the like; or mixtures thereof. The concentration of rosiglitazone maleate in the solvent can range from about 10 to about 80% or more. For recrystallization, a solution can be prepared at an elevated temperature if desired to achieve a desired concentration. Any temperature is acceptable for the dissolution as long as a clear solution of the rosiglitazone maleate is obtained and is not detrimental to the drug substance chemically or physically. The solution may be brought down to room temperature for further processing if required or an elevated temperature may be used. A higher temperature will allow the precipitation from solutions with higher concentrations of rosiglitazone maleate resulting in better economies of manufacture. Drying can be carried out until the residual solvent content reduces to within the limits given by the ICH guidelines. The solvent level depends on the type of solvent used, but generally, it is not more than about 5000 ppm, or about 4000 ppm, or about 3000 ppm.

The purified rosiglitazone maleate obtained using the above process contains less than 0.15 area-% or less than 0.05 area-% of the rosiglitazone succinic acid impurity. The purified rosiglitazone maleate obtained using the above process contains less than about 1000 ppm, or less than about 50 ppm of methanol, acetone, isopropyl alcohol, dichloromethane, toluene, acetic acid, and N,N dimethylformamide.

In another embodiment, the present invention provides a process for the preparation of rosiglitazone succinic acid impurity which comprises reacting rosiglitazone maleate with water. Suitable temperatures for conducting the reaction range from about 20° C. to about 200° C. or from about 50° C. to about 100° C. Suitably, no other additional solvent is used and the reaction is conducted using water as the solvent medium.

Regulatory authorities require declarations that the active agent is acceptable for administration to humans and that the particular formulation, which is to be marketed, is free from impurities at the time of release and has an appropriate shelf life. While submitting these declarations, drug manufacturers must include analytical records to demonstrate that impurities are absent from the drug at the time of manufacture, or are present only at a negligible level, and that the storage stability, i.e., shelf-life of the drug is acceptable.

These details are usually obtained by testing the drug against an external standard, or reference marker, which is a pure sample of a potential impurity or a potential degradation product. Rosiglitazone succinic acid impurity is useful as a reference marker compound in identifying the purity of the rosiglitazone maleate.

A compound in a relatively pure state can be used as a “reference standard.” A reference standard is similar to a reference marker, which not only is used for qualitative analysis, but is also used to quantify the amount of the compound of the reference standard in an unknown mixture, as well. The management of process impurities is greatly enhanced by understanding their chemical structures and synthetic pathways, and by identifying the parameters that influence the amount of impurities in the final product. The detection or quantification of the reference standard serves to establish the level of purity of the API or intermediates thereof. Use of a compound as a standard requires recourse to a sample of the substantially pure compound.

In an embodiment the present invention provides 2-[5-[[4-[2-(methyl-2-pyridinylamino)ethoxy]phenyl]methyl]-2,4-thiazolidinedione] succinic acid of Formula II, which is useful as a reference standard for determining the purity of rosiglitazone by HPLC, and a process for its preparation.

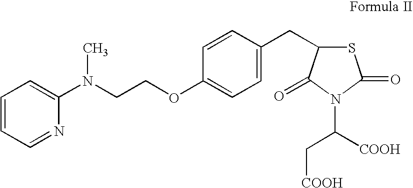

### The structure of the molecule has been confirmed by H¹ NMR, Mass and IR spectrum.

Optionally, the 2-[5-[[4-[2-(methyl-2-pyridinylamino)ethoxy]phenyl]methyl]-2,4-thiazolidinedione]succinic acid of Formula II obtained from the reaction can be further purified by recrystallization or slurry in a suitable solvent.

Another embodiment of the present invention provides a pharmaceutical composition that includes a therapeutically effective amount of pure rosiglitazone and its salts prepared according to the processes of the present invention and one or more pharmaceutically acceptable carriers, excipients or diluents.

The pharmaceutical composition comprising substantially pure rosiglitazone or its pharmaceutically acceptable salts along with one or more pharmaceutically acceptable carriers of this invention may further formulated as: solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared by direct blending, dry granulation or wet granulation or by extrusion and spheronization. Compositions may be presented as uncoated, film coated, sugar coated, powder coated, enteric coated or modified release coated. Compositions of the present invention may further comprise one or more pharmaceutically acceptable excipients.

Pharmaceutically acceptable excipients that find use in the present invention include, but are not limited to: diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinyl pyrrolidone, hydroxypropyl cellulose, hydroxypropyl methylcellulose, pregelatinized starch and the like; disintegrants such as starch, sodium starch glycolate, pregelatinized starch, crospovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins, resins; release rate controlling agents such as hydroxypropyl cellulose, hydroxymethyl cellulose, hydroxypropyl methylcellulose, ethyl cellulose, methyl cellulose, various grades of methyl methacrylates, waxes and the like Other pharmaceutically acceptable excipients that are of use include but are not limited to film formers, plasticizers, colorants, flavoring agents, sweeteners, viscosity enhancers, preservatives, antioxidants and the like.

In one embodiment of the present invention, rosiglitazone or its pharmaceutically acceptable salts is a useful active ingredient and is present in the range of from about 0.5 mg to about 15 mg, or from about 1 mg to about 10 mg.

Certain specific aspects and embodiments of this invention are described in further detail by the examples below, which examples are provided only for the purpose of illustration and are not intended to limit the scope of the appended claims in any manner.

### EXAMPLE 1

### Purification of Crude Rosiglitazone from Its Related Dehydro Rosiglitazone Impurity

35 ml of SEPABEADS SP207SS (Mitsubishi chemical corporation, Japan) were washed with acetone followed by water and packed in 1.1 cm×40 cm borosilicate glass column fitted with BioRad (USA) Econo adaptors at both the end. The top of the column was connected to Biologic Duoflow chromatographic system from BioRad, USA. The outlet of the column was connected to the QuadTec UVNIS detector so as to observe the column performance. The resin column was then irrigated with 2.5 column volumes of 5.0 Mm potassium dihydrogen phosphate buffer in distilled water and the pH adjusted to 2.8 with 30% v/v solution of ortho-phosphoric acid.

1.2 gm of crude rosiglitazone containing about 0.24% w/w dehydro rosiglitazone impurity was dissolved in 35 ml of distilled water with the aid of about 30% v/v solution of ortho-phosphoric acid and the volume was adjusted to 40 ml with distilled water. This rosiglitazone solution was then loaded in downward direction to the above column at flow rate of 2 ml/min using the above chromatographic system followed by washing with an irrigating phosphate buffer. Elution of adsorbed rosiglitazone was carried out using 0% v/v to 80% v/v gradient of methanol in an irrigating phosphate buffer in 1.8 column volumes. In this mobile phase, rosiglitazone was retained less strongly than dehydro rosiglitazone and it therefore eluted out first. The fractions corresponding to rosiglitazone peak were collected and analyzed by HPLC. The fractions showing pure rosiglitazone were pooled and evaporated to dryness. The solid obtained after evaporation was again analyzed by HPLC and Mass spectroscopy.

% Yield: 97.8%.

Purity By HPLC: 99.85%.

% of dehydro rosiglitazone impurity: Below LOD. (LOD: 0.0003)

### EXAMPLE 2

### Purification of Purification of Rosiglitazone from Its Related Dehydro Rosiglitazone Impurity Under Different Gradient Volumes

2 gm of crude rosiglitazone maleate was dissolved in 40 ml of distilled water with the aid of ortho-phosphoric acid (25% v/v solution in water). The rest of the process was operated under different gradient volumes using two different adsorbent matrices as given in Table 1:

TABLE 1

| | | | | HPLC | |
|---|---|---|---|---|---|
| | | Gradient | | purity | Impurity |
| | | volume | Recovery | (% | level |
| S. No. | Matrix used | (CV) | (% w/w) | area) | % w/w |
| 1 | SEPABEADS | 0.7 | 74.2 | 99.68 | Below LOD |
| | SP207SS | | | | |
| 2 | SEPABEADS | 1.5 | 80.0 | 99.84 | Below LOD |
| | SP207SS | | | | |
| 3 | SEPABEADS | 2.0 | 96.9 | 99.86 | Below LOD |
| | SP207SS | | | | |
| 4 | SEPABEADS | 10 | 94.8 | 98.86 | Below LOD |
| | SP207SS | | | | |
| 5 | SEPABEADS | 0.7 | 64.3 | 98.2 | Below LOD |
| | HP20SS | | | | |
| 6 | SEPABEADS | 1.5 | 76.8 | 99.2 | Below LOD |
| | HP20SS | | | | |
| 7 | SEPABEADS | 2.0 | 82.4 | 99.1 | Below LOD |
| | HP20SS | | | | |

### EXAMPLE 3

### Purification of Crude Rosiglitazone from Its Related Impurity

1.2 liter of SEPABEADS SP207SS (Mitsubishi Chemical Corporation, Japan) were washed with acetone followed with water and packed in a column with 5.0 cm diameter and length 1.2 meter. The column was connected to the same chromatographic system as described in Example 1. 18 gm of crude rosiglitazone containing 1.5% w/w dehydro rosiglitazone impurity was dissolved in 400 ml of 5 mM phosphate buffer, pH 2.8 with aid of ortho-phosphoric acid (30% v/v solution). The resin column was equilibrated with 2 column volumes of phosphate buffer until the outlet conductivity was 1.6 mS/cm. The rosiglitazone crude solution was passed through the column at a flow rate of 30 ml/min in a downward direction using a peristaltic pump followed by washing with a 1 column volume of phosphate buffer, pH 2.8. The adsorbed rosiglitazone was then eluted using 5% v/v to 70% v/v linear gradient of methanol in phosphate buffer in 2 column volumes at a flow rate of 65 ml/min followed by isocratic elution with 70% v/v methanol in a phosphate buffer. The elution fractions were collected and distilled under vacuum to recover the solid rosiglitazone. The percentage of dehydro rosiglitazone impurity in the recovered sample was below the limit of detection on HPLC.

% Yield: 95%.

Purity by HPLC: 99.65%.

### EXAMPLE 4

### Regeneration and Reuse of Chromatographic Matrix for Purification of Rosiglitazone from Its Related Impurity

After elution of rosiglitazone from the adsorbed matrix, the matrix of Example 3 was regenerated with 3 column volumes of a mixture of 50:50 compositions of acetonitrile and acetone at 1.0 bed volume per hour flow rate. The regenerated matrix was again reused according to the process in Example 3. The results obtained from three consecutive trials showed recovery of more than 95% and purity of more than 99% area on HPLC for rosiglitazone with related impurity below the limit of detection.

### EXAMPLE 5

### Purification of Rosiglitazone from Its Related Impurity on ODS Silica

70 ml of ODS silica gel was packed in 2.1 cm×25 cm stainless steel column. The resin column was irrigated with 2 column volumes of 10 mM solution of sodium phosphate, pH 3.1. 1.2 gm of crude rosiglitazone containing 0.14% w/w dehydro rosiglitazone was dissolved in distilled water with the aid of 25% v/v solution of ortho-phosphoric acid and loaded onto the column followed by washing with irrigating buffer. Elution of adsorbed rosiglitazone was performed with 80% v/v solution of acetonitrile in the above buffer at 8 ml/min flow rate. 97% of rosiglitazone was recovered showing related impurity below the limit of detection.

Purity by HPLC: 99.94%.

### EXAMPLE 6

### Preparation of Rosiglitazone Maleate (Formula I)

44 liters of acetone was charged into a clean reactor and 5.5 kg of rosiglitazone freebase was added to it. The reaction mass was heated to about 60° C. and 1.82 kg of a solution of maleic acid in 9 liters of acetone was added. 0.55 kg of activated carbon was added and the reaction mass was stirred for about 25 minutes. The reaction suspension was filtered through celite and the filtered bed was washed with 28 liters of acetone. The combined filtrate was cooled to 30° C. and the solvent was partially distilled at a temperature of about 30° C. under a vacuum of 550 mm/Hg. The resultant reaction mass was cooled to a temperature of about 2.5° C. with simultaneous stirring, and maintained for about 45 minutes. The separated solid was filtered and the wet solid was dried at about 30° C. for about 12 hours to afford the rosiglitazone maleate of Formula I.

Purity by HPLC: 99.6%.

% of dehydro rosiglitazone impurity: 0.01%.

% of Succinic acid impurity: Less than 0.03%.

Residual Solvent Content: Acetone: 225 ppm.

All other residual organic solvents: Less than 20 ppm.

### EXAMPLE 7

### Preparation of2-[(5-[4-[2-Methyl (2-Pyridyl)-Amino Ethoxy]Benzyl]-2,4-Dioxo-1,3-Thiazolan-3-YL)]Succinicacid (Formula II)

10 g of rosiglitazone maleate and 2 ml of water were taken into a clean and dry round bottom flask and heated to about 95° C. The mixture was maintained at about 95° C. for about 148 hours to afford 8.4 g of crude compound of Formula II.

8.0 g of the crude obtained and 16 ml of methanol were taken into a clean and dry round bottom flask arranged in a water bath. The reaction mass was heated to about 65° C. and maintained for a period of about 45 minutes followed by cooling to about 40° C. with simultaneous stirring. The separated solid was filtered and washed with 8 ml of methanol followed by suction drying. The solid obtained was dried at about 55° C. for about 3 hours to afford 3.9 g of the title compound of Formula II.

Purity by HPLC: 94.9%.

IR: 3547 (O—H), 3043 (Ar—CH), 2943 (Ali C—H), 1755, 1715 (—C═O), 1688(—C═O)₂, 1180 (—C—N) and 764 (Ar C—H bending).

1H NMR: (DMSO-d6, 400 MHz): δ 4.97 (dd, 1H J=4.8, 9.2), δ 7.15 (d, 1H J=8.8), δ6.86 (d, 1H J=8.4), δ4.11 (t, 2H, J=5.8), δ 3.89 (t,1 H J=5.8), δ 3.06 (s, 3H), δ 8.07 (d, 1H J=6.4), δ 6.56 (t,1H J=6.4), δ 7.5 (t, 1H J=8.8), δ 6.65 (d, 1H J=8.8), δ 5.07 (t 1H J=6.4).

MS: m/z 474 (100%, M+1)

### EXAMPLE 8

### Tablet Compositions of Rosiglitazone Maleate Prepared from Example 6:

### Composition:

| | | 2 mg | 4 mg | |
|---|---|---|---|---|
| | | tablets | tablets | 8 mg tablets |
| S. No. | Material | (mg/tab) | (mg/tab) | (mg/tab) |
| 1 | Rosiglitazone maleate | 2.65 | 5.3 | 10.6 |
| 2 | Lactose monohydrate | 46.85 | 93.7 | 187.4 |
| 3 | Microcrystalline | 15 | 30 | 60 |
| | cellulose | | | |
| 4 | Sodium starch glycolate | 6 | 12 | 24 |
| 5 | Pregelatinized starch | 3.75 | 7.5 | 15 |
| 6 | Purified water | Q.S | Q.S | Q.S |
| 7 | Magnesium stearate | 0.75 | 1.5 | 3 |
| 8 | Opadry White OY- | 2 | 4 | 8 |
| | 58900* | | | |

*Supplied by Colorcon Asia pvt. Limited.

### Manufacturing Process:

1. Materials 1-5 were sifted through 40# ASTM sieve.
2. Sifted materials 1-3 and half the quantity of material 4 were blended in a rapid mixer granulator and granulated with purified water, granules were dried in a fluid bed granulator till the loss on drying of the granules was less than 2%.
3. Dried granules were sifted through 20# ASTM sieve and blended with 2^{nd} half of material 4 and magnesium stearate (sifted through 60# ASTM sieve) in a double cone blender.
4. Lubricated blend from step 3 is compressed to get 2 mg or 4 mg or 8 mg tablets adjusting the weights to 75 mg or 150 mg or 300 mg respectively.

Compressed tablets were coated using Opadry white OY-58900 water dispersion till the final tablet weight is 77 mg or 154 mg or 308 mg for 2 mg or 4 mg or 8 mg tablets respectively.

1. A composition comprising:
(a) a compound which is rosiglitazone maleate of Formula I 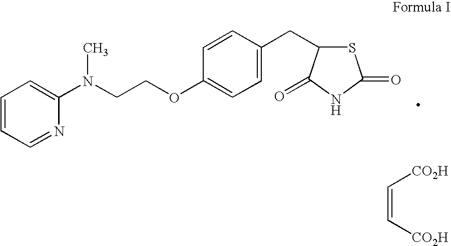
present in the amount of about 05 mg to about 15 mg; and
(b) a compound which is rosiglitazone succinic acid of Formula II, 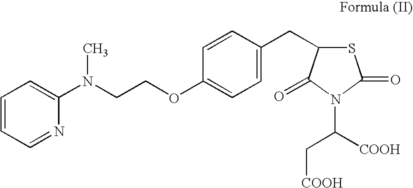
present in an amount from about 0.001% to not more than about 0.15 area percent by HPLC

2. The composition of claim 1, wherein said rosiglitazone maleate of Formula I is present in the amount of about 1 mg to about 10 mg.

3. The composition of claim 1, wherein said rosiglitazone succinic acid of Formula II, is present in an amount of from about 0.001% to not more than about 0.05 area percent by HPLC

4. The composition of claim 1, wherein said rosiglitazone succinic acid of Formula II, is present in an amount of from about 0.001% to not more than about 0.10 area percent by HPLC

5. A composition comprising:
(a) a compound which is rosiglitazone maleate of Formula I, present in the amount of about 05 mg to about 15 mg; and
(b) a compound which is the dehydro impurity of rosiglitazone having a Formula III: 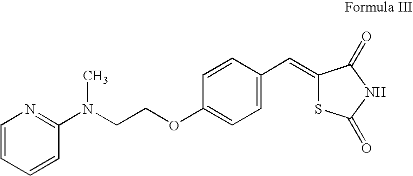
present in an amount of about 0.001% to not more than about 0.10 area percent.

6. The composition of claim 5, wherein said rosiglitazone maleate of Formula I is present in the amount of about 1 mg to about 10 mg.

7. A composition comprising
(a) a compound which is rosiglitazone maleate of Formula I present in the amount of about 05 mg to about 15 mg;
(b) a compound which is rosiglitazone succinate of Formula II, present in an amount of about 0.001% to not more than about 0.15 area percent by HPLC; and
(c) a compound which is the dehydro impurity of rosiglitazone having a Formula III present in an amount of about 0.001% to not more than about 0.10.

8. The composition of claim 7, wherein said rosiglitazone maleate of Formula I is present in the amount of about 1 mg to about 10 mg.

9. The composition of claim 7, wherein said rosiglitazone succinate of Formula II, is present in an amount of about 0.001% to not more than about 0.05 area percent by HPLC.

10. The composition of claim 7, wherein said rosiglitazone succinic acid of Formula II, is present in an amount of from about 0.001% to not more than about 0.10 area percent by HPLC

11. A process for the preparation of rosiglitazone maleate of Formula I, wherein rosiglitazone succinate impurity of Formula II is present in an amount of about 0.001% to not more than 0.15 area percent by HPLC, which process comprises:
(a) heating a reaction mass containing rosiglitazone freebase and a ketone to about 60° C.
(b) adding maleic acid dissolved in an organic solvent followed by addition of activated carbon;
(c) isolating rosiglitazone maleate;
(d) drying at temperatures of from about 25° C. to about 40° C. with or without vacuum and with or without inert atmosphere, to afford the desired rosiglitazone maleate compound of desired purity.

12. The process of claim 11, wherein said rosiglitazone succinate of Formula II, is present in an amount of less than about 0.001% and not more than about 0.10 area percent by HPLC

13. The process of claim 11, wherein said rosiglitazone succinic acid of Formula II, is present in an amount of from about 0.001% to not more than about 0.05 area percent by HPLC

14. Rosiglitazone succinic acid of Formula II

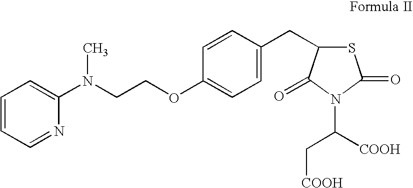

15. A process for preparing the rosiglitazone succinic acid of claim 14, which process comprises heating rosiglitazone maleate in water.

16. A process for the preparation of rosiglitazone maleate of Formula I, wherein rosiglitazone succinate impurity of Formula II is present in an amount of about 0.001% to not more than about 0.15% by HPLC, which process comprises:
a) providing a solution of rosiglitazone in a suitable solvent;
b) adding maleic acid to the solution obtained in step (a);
c) isolating rosiglitazone maleate from the solution of step b); and
d) drying the solid obtained at a temperature of about 40° C.

17. The process of claim 16, wherein drying in step d) is carried out at a temperature of about 30° C.

18. The process of claim 16, wherein said rosiglitazone succinate impurity of Formula II is present in an amount of about 0.001% to not more than about 0.10% by HPLC.

19. The process of claim 16, wherein said rosiglitazone succinate impurity of Formula II is present in an amount of about 0.001% to not more than about 0.05% by HPLC.

20. A process for purifying rosiglitazone, comprising:
a) adsorbing rosiglitazone onto an adsorbent resin, by contacting a resin with a rosiglitazone solution; and
b) eluting purified rosiglitazone from a resin with a solvent comprising 5% v/v to 70% v/v linear gradient of methanol in phosphate buffer.

21. The process of claim 20, wherein an adsorbent resin comprises pre-equilibrated SEPABEADS SP207SS.

22. Pharmaceutical composition comprising rosiglitazone or its pharmaceutically acceptable salts free of at least one of the dehydro and the succinic acid impurities of rosiglitazone along with one or more pharmaceutically acceptable carriers, excipients or diluents.
